SEQUENCE LISTING (14 sequences)

SEQ ID NO: 1 - protein, 85 aa, S. cerevisiae
MRFPSIFTAVLFAASSALAAPVNTTTEDETAQIPAEAVIGYSDLEGDFDVAVLPFSNSTNNGLLFINTTIASIAAKEEGVSMAKR

SEQ ID NO: 2 - protein, 6 aa, Artificial Sequence
  Synthetic
EEGEPK

SEQ ID NO: 3 - DNA, 39 bp, Artificial Sequence
  Synthetic
atacaggaattccattcaagatctgttcaaacaagaaga

SEQ ID NO: 4 - DNA, 106 bp, Artificial Sequence
  Synthetic
aatcttagtttctagactagttgcagtagttttccaattggtacaaggagcagatggaggtacagcattgttcgacaatacccttmnnagccttaggagtgtagaa

SEQ ID NO: 5 - DNA, 432 bp, Artificial Sequence
  Synthetic
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatccatggctaagagagaagaaggtgaaccaaagttcgttaaccaacacttgtgcggttcccacttggttgaagctttgtacttggtttgcggtgaaagaggtttcttctacactcctaaggctgctaagggtattgtcgaacaatgctgtacctccatctgctccttgtaccaattggaaaactactgcaac

SEQ ID NO: 6 - protein, 144 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSMAKREEGEPKFVNQHLCGSHLVEALYLVCGERGFFYTPKAAKGIVEQCCTSICSLYQLENYCN

SEQ ID NO: 7 - protein, 85 aa, Artificial Sequence; identical to SEQ ID NO: 1
  Synthetic

SEQ ID NO: 8 - protein, 66 aa, Artificial Sequence
  Synthetic
MKLKTVRSAVLSSLFASQVLGQPIDDTESNTTSVNLMADDTESRFATNTTLAGGLDVVNLISMAKR

SEQ ID NO: 9 - DNA, 432 bp, Artificial Sequence
  Synthetic
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatccatggctaagagagaagaaggtgaaccaaagttcgttaaccaacacttgtgcggttcccacttggttgaagctttgtacttggtttgcggtgaaagaggtttcttctacactcctaaggctnnkaagggtattgtcgaacaatgctgtacctccatctgctccttgtaccaattggaaaactactgcaac

SEQ ID NO: 10 - protein, 144 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSMAKREEGEPKFVNQHLCGSHLVEALYLVCGERGFFYTPKAXKGIVEQCCTSICSLYQLENYCN

SEQ ID NO: 11 - DNA, 375 bp, Artificial Sequence
  Synthetic
atgaaactgaaaactgtaagatctgcggtcctttcgtcactctttgcatctcaggtccttggccaaccaattgacgacactgaatctaacactacttctgtcaacttgatggctgacgacactgaatccagattcgctactaacactactttggctggtggtttggatgttgttaacttgatctccatggctaagagagaagaaggtgaaccaaagttcgttaaccaacacttgtgcggttcccacttggttgaagctttgtacttggtttgtggtgaaagaggtttcttctacactcctaaggctatgaagggtattgtcgaacaatgctgtacctccatctgctccttgtaccaattggaaaactactgcaac

SEQ ID NO: 12 - protein, 125 aa, Artificial Sequence
  Synthetic
MKLKTVRSAVLSSLFASQVLGQPIDDTESNTTSVNLMADDTESRFATNTTLAGGLDVVNLISMAKREEGEPKFVNQHLCGSHLVEALYLVCGERGFFYTPKAMKGIVEQCCTSICSLYQLENYCN

SEQ ID NO: 13 - protein, 85 aa, S. cerevisiae; identical to SEQ ID NO: 1

SEQ ID NO: 14 - protein, 85 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLDKR

(19) United States
(12) Patent Application Publication
(10) Pub. No.: US 2016/0115216 A1
(43) Pub. Date: Apr. 28, 2016

(54) Method for Making Mature Insulin Polypeptides

(71) Applicant: NOVO NORDISK A/S, Bagsværd (DK)

(72) Inventors: Frantisek Hubalek, Herlev (DK); Annette F. Pettersson, Farum (DK); Thomas B. Kjeldsen, Virum (DK); Asser S. Andersen, Herlev (DK)

(21) Appl. No.: 14/895,741

(22) PCT Filed: Jun. 6, 2014
(86) PCT No.: PCT/EP2014/061812

§ 371 Date: Dec. 3, 2015

Related U.S. Application Data
(60) Provisional application No. 61/833,613, filed on Jun. 11, 2013.

(30) Foreign Application Priority Data
Jun. 7, 2013 (EP) 13171029.5

(51) Int. Cl. C07K 14/62

(52) U.S. Cl. CPC C07K 14/62

(57) ABSTRACT

This invention relates to an improved method for making mature human insulin or an analogue thereof by cultivating fungi cell comprising a DNA sequence encoding a precursor for human insulin or an analogue thereof, which precursor comprises a small connecting peptide.

### TECHNICAL FIELD

The present invention relates to recombinant protein expression and protein chemistry where mature insulin polypeptides are made.

### BACKGROUND

Insulin is a polypeptide hormone produced in the beta cells of the islets of Langerhans. The active insulin molecule is a two-chain molecule consisting of a B- and an A-chain connected by two disulphide bridges. The insulin is synthesized as a precursor molecule proinsulin with the structure B-C-A wherein the C-peptide chain connects the C-terminal amino acid residue in the B-chain with the N-terminal amino acid residue in the A-chain. Mature two-chain insulin is formed by in vivo cleavage of the C-peptide at the pair of basic amino acid residues situated at the junction with the A- and B-chain. The A- and B-chain are held together by two disulphide bridges between the A7 and B7 and the A20 and B19 Cys residues, respectively. In addition, the biologically active insulin molecule has an internal disulphide bridge between the Cys residues in the positions A6 and A11.

A number of methods have been described to produce insulin and precursors thereof in genetically modified host cells, such as E. coli and yeasts. In most of the yeast processes a precursor of insulin with either the natural C-peptide or with a modified C-peptide are expressed and secreted from the yeast cell. WO90/10075 discloses an insulin precursor having the C-peptide AAK. WO01/49742 discloses insulin precursors having a C-peptide comprising an aromatic amino acid residue. WO02/079251 discloses insulin precursors having a C-peptide comprising a Gly residue. WO02/079250 discloses insulin precursors having a C-peptide comprising a Pro residue. WO02/100887 discloses insulin precursors having a C-peptide comprising a glycosylation site. WO2008/037735 discloses insulin precursors having a C-peptide comprising a kex2p cleavage site. WO2011/099028 discloses a method for reducing O-glycosylation levels of the insulin or insulin analog precursor molecule produced in Pichia sp.

If the mature insulin or insulin analogue product is not directly obtained it is then obtained in one or more subsequent in vitro enzymatic steps by cleavage of the C-peptide and possibly the N-terminal extension. These enzymatic steps are time consuming and often tend to be costly as well as posing the risk of introducing additional related impurities, i.e. impurities resembling the maturated insulin polypeptide. Another challenge of yeast expression of insulin polypeptides is O-glycosylation of the insulin polypeptides. O-glycosylated insulin polypeptides are also related impurities. Common to all the related impurities is that they are technically difficult and thus expensive to remove in commercial purification processes. This is due to the requirement for extra purification steps, typically chromatography steps, or the requirement for chromatography steps operating at economically unfavourable conditions. Such chromatography steps may operate at longer cycle time, lower column load, or even lower yield as a consequence of the related impurities.

In the pharmaceutical industry insulin products increasingly are constituted by derivatives of insulin polypeptides and increasingly such drug products are for non-injectable delivery. Hence, the insulin market already being a competitive market and moving towards products requiring more insulin polypeptides per dosage, there is a need for more cost-effective processes for making insulin polypeptides.

Thus, there is a need for making human insulin or analogues thereof by fungi in an industrial process having reduced O-glycosylation of the insulin precursor molecule. There is also a need for industrial processes having higher yields of insulin precursor as well as insulin precursors amenable for proteolytic excision of the C-peptide by an effective and simple process.

### SUMMARY

The present invention provides novel connecting peptides (C-peptides) which confer high yield of insulin precursor molecules when expressed in a transformed microorganism, in particular yeast. The novel connecting peptides also facilitate a generally low level of O-glycosylation when expressed in fungi, such as yeast. Expressing the novel insulin precursors in fungi strain having reduced capacity for O-glycosylation further decreases the proportion of the expressed insulin precursors which are O-glycosylated. Such insulin precursors can then be converted into human insulin, desB30 human insulin, other insulin analogues or certain insulin derivatives by one or more suitable, well known conversion steps.

According to a first aspect of the invention, there is provided a method for making mature human insulin or an analogue thereof by culturing a fungi cell comprising a DNA sequence encoding a precursor for human insulin or an analogue thereof with the sequence Z-B-X-Y-A wherein
Z is an optional extension sequence,
B is the B-chain of human insulin or an analogue thereof,
X is a sequence selected from X₁M, EA, AE, AD, DA, and AP, where X₁ is a sequence comprising from 1 to 3 amino acid residues,
Y is K or R, and
A is the A-chain of human insulin or an analogue thereof.

According to a second aspect of the invention there is provided an insulin precursor comprising the sequence Z-B-X-Y-A wherein
Z is an optional extension sequence,
B is the B-chain of human insulin or an analogue thereof,
X is a sequence selected from X₁M, EA, AE, AD, DA, and AP, where X₁ is a sequence comprising from 1 to 3 amino acid residues,
Y is K or R, and
A is the A-chain of human insulin or an analogue thereof.

According to a third aspect of the invention there is provided a method for reducing O-glycosylation of a precursor for human insulin or an analogue of human insulin during expression in a fungi cell, said method comprising (i) culturing a fungi cell comprising a DNA sequence encoding a precursor for human insulin or an analogue thereof with the sequence Z-B-X-Y-A wherein
Z is an optional extension sequence,
B is the B-chain of human insulin or an analogue thereof,
X is a sequence selected from X₁M, EA, AE, AD, DA, and AP, where X₁ is a sequence comprising from 1 to 3 amino acid residues,
Y is K or R, and
A is the A-chain of human insulin or an analogue thereof, under suitable culture conditions for expression of said precursor for human insulin or an analogue of human insulin.

According to a fourth aspect of the invention there is provided a method for increasing the yield of a precursor for human insulin or an analogue of human insulin during expression in a fungi cell, said method comprising (i) culturing a fungi cell comprising a DNA sequence encoding a precursor for human insulin or an analogue thereof with the sequence Z-B-X-Y-A wherein
Z is an optional extension sequence,
B is the B-chain of human insulin or an analogue thereof,
X is a sequence selected from X₁M, EA, AE, AD, DA, and AP, where X₁ is a sequence comprising from 1 to 3 amino acid residues,
Y is K or R, and
A is the A-chain of human insulin or an analogue thereof,
   under suitable culture conditions for expression of said precursor for human insulin or an analogue of human insulin.

The methods of the present invention provide a number of advantages over previously described methods for making mature human insulin or an analogue thereof by culturing a fungi cell. For example, it has been found that the insulin precursors according to the present invention are expressed in fungi in very high yields. The novel insulin precursors have surprisingly been found also to cause low amounts of related impurities in the form of O-glycosylated insulin precursors. It has further been discovered that the low amounts of O-glycosylated insulin precursors can be reduced even further to 2-4 fold by using different protein mannosyl transferase knock-out strains. Hence, the objective is to provide insulin precursors exhibiting high expression levels in fungi as well as the expressed insulin precursors having a low level of O-glycosylation. Since, it has surprisingly been found that O-glycosylation levels may be reduced both by the selection of the C-peptide in the insulin precursor as well as by the use of PMT modulated strains, high expression yields remain important.

Lower O-glycosylation allows for optimization of the up-stream fermentation process as well as simultaneous optimization of the down-stream conversion and purification processes where any O-glycosylated forms must be finally eliminated. The insulin precursors of the invention furthermore facilitate an efficient maturation by cleavage by proteases, e.g. Acromobacter lyticus protease (ALP). Hence, the result of this combined optimization of fermentation yield, O-glycosylation and ALP cleavage allows for significantly higher fermentation yield, significantly higher loads on purification columns and even elimination of purification steps from currently used processes as well as a streamlined ALP cleavage reaction step. The resulting overall process therefore increases capacity of manufacturing plants while at the same time reducing the amount of raw materials needed to produce the insulin polypeptides. Both of these results drive down the cost of the insulin polypeptides.

The fungi cell used as host cell for expressing the precursor for human insulin or an analogue of human insulin may carry at least one genetic modification reducing its capacity for O-glycosylation. The connecting peptide, X-Y, of the invention causes a low O-glycosylation of the insulin precursors excreted from the fungi cell. However, for some of the C-peptides an even lower level of O-glycosylation is obtained by expression in a fungi cell having reduced capacity for O-glycosylation. In one embodiment, said genetic modification reducing the fungi cells capacity for O-glycosylation is at least one genetic modification within the genes for PMT1 or PMT2.

In one embodiment of the present invention the connecting peptide, X-Y in the sequence Z-B-X-Y-A is X₁M-Y, where X₁ is a sequence comprising from 1 to 3 amino acid residues. Accordingly, in one aspect the present invention relates to insulin precursors comprising a connecting peptide (X-Y) being cleavable from the A and B chains and comprising at least one M and a cleavage site enabling cleavage of the peptide bond between the A-chain and the connecting peptide, wherein one M is immediately N-terminal to said cleavage site.

In another aspect the present invention relates to insulin precursors comprising a connecting peptide (C-peptide) being cleavable from the A and B chains and consisting of from 3 to 5 amino acid residues of which at least one is a M residue.

The present invention is also related to polynucleotide sequences which code for the claimed insulin precursors. In a further aspect the present invention is related to vectors containing such polynucleotide sequences and host cells containing such polynucleotide sequences or vectors.

### BRIEF DESCRIPTION OF DRAWINGS

FIG. 1 shows the pAK1119 S. cerevisiae expression plasmid expressing the alpha*-leader (without the BgIII-site) (SEQ ID NO:1)-EEGEPK (SEQ ID NO:2)-insulin precursor fusion protein.

FIG. 2 shows the nucleotide sequence of pAK1119 DNA expression cassette (SEQ ID NO:5) and inferred amino acids of the encoded fusion protein (alpha*-leader-EEGEPK-insulin precursor of pAK1119 (SEQ ID NO:6).

FIG. 3 shows the pAK3768 S. cerevisiae expression plasmid expressing the alpha2-leader-EEGEPK-B(1-29)-AlaXLys-A(1-21) precursor.

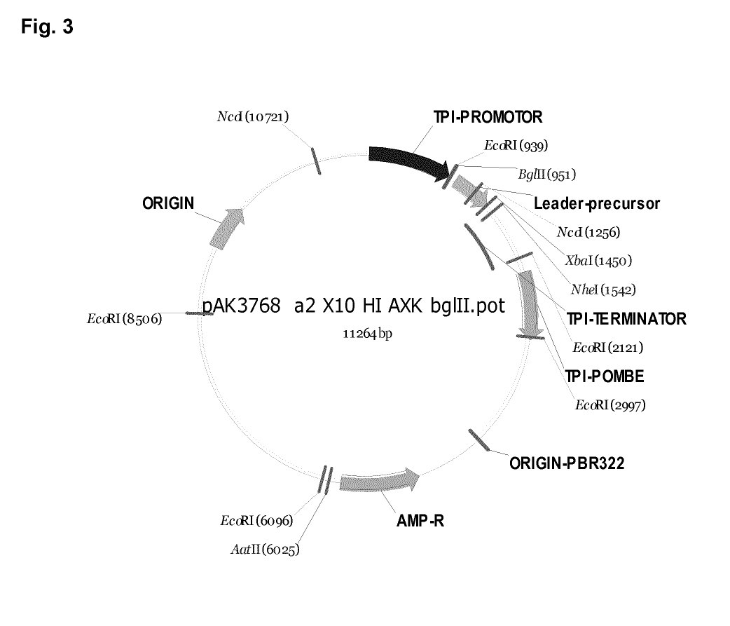

FIG. 4 shows the nucleotide sequence of pAK3768 DNA expression cassette (SEQ ID NO:9) and inferred amino acids of the encoded fusion protein (alpha2-leader-EEGEPK-insulin precursor of pAK3768 (SEQ ID NO:10).

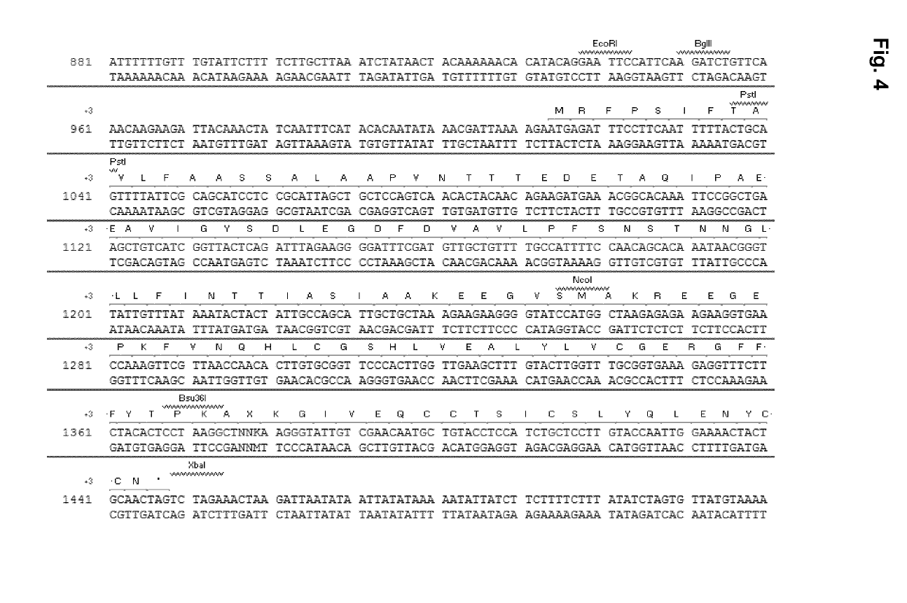

FIG. 5 shows the pAK4053 S. cerevisiae expression plasmid expressing the TA39-leader-EEGEPK-B(1-29)-AlaMetLys-A(1-21) precursor.

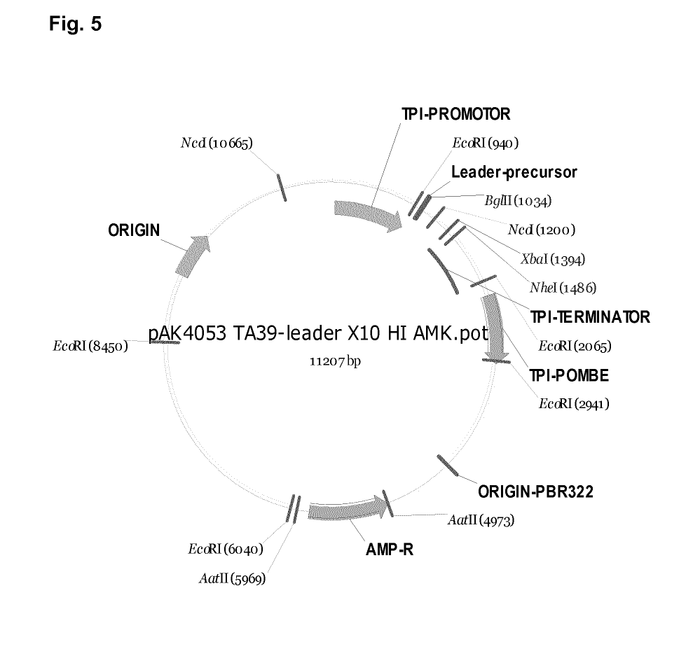

FIG. 6 shows the nucleotide sequence of pAK4053 DNA expression cassette (SEQ ID NO:11) and inferred amino acids of the encoded fusion protein (TA39-leader-EEGEPK-insulin precursor of pAK4053 (SEQ ID NO:12)).

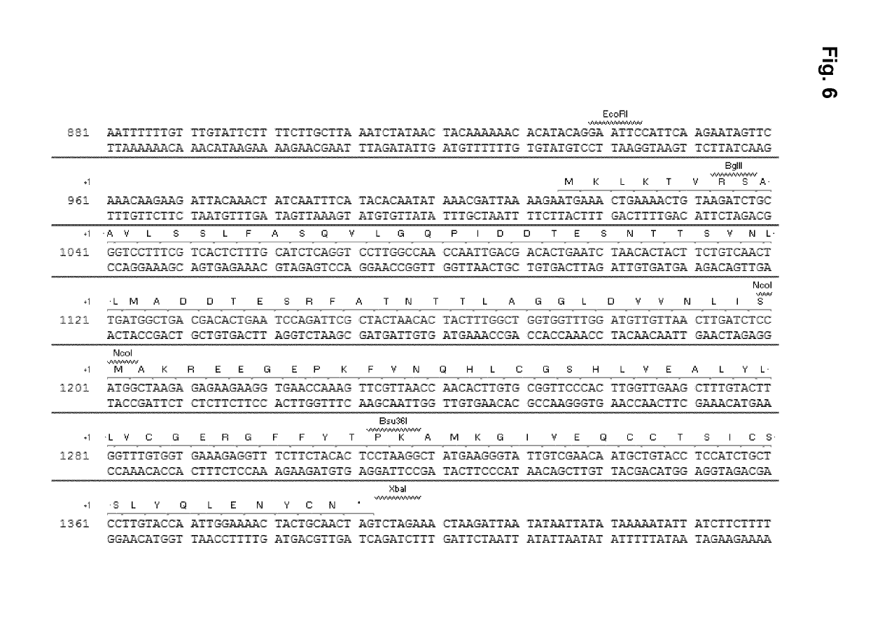

### DESCRIPTION

According to a first aspect of the invention, there is provided a method for making mature human insulin or an analogue thereof by culturing a fungi cell comprising a DNA sequence encoding a precursor for human insulin or an analogue thereof with the sequence Z-B-X-Y-A wherein
Z is an optional extension sequence,
B is the B-chain of human insulin or an analogue thereof,
X is a sequence selected from X₁M, EA, AE, AD, DA, and AP, where X₁ is a sequence comprising from 1 to 3 amino acid residues,
Y is K or R, and
A is the A-chain of human insulin or an analogue thereof.

The term “insulin analogue” as used herein means a modified human insulin wherein one or more amino acid residues of the insulin have been substituted by other amino acid residues and/or wherein one or more amino acid residues have been deleted from the insulin and/or wherein one or more amino acid residues have been added and/or inserted to the insulin. The insulin analogues will typically not comprise more than about 7 mutations, more typically not more than 5 and even more typically at the most 3 mutations compared to human insulin. In one embodiment an insulin analogue comprises less than 10 amino acid modifications (substitutions, deletions, additions (including insertions) and any combination thereof) relative to human insulin, alternatively less than 9, 8, 7, 6, 5, 4, 3, 2 or 1 modification relative to human insulin.

Modifications in the insulin molecule are denoted stating the chain (A or B), the position, and the one letter code for the amino acid residue substituting the native amino acid residue. Herein terms like “A1”, “A2” and “A3” etc. indicates the amino acid in position 1, 2 and 3 etc., respectively, in the A chain of insulin (counted from the N-terminal end). Similarly, terms like B1, B2 and B3 etc. indicates the amino acid in position 1, 2 and 3 etc., respectively, in the B chain of insulin (counted from the N-terminal end). Using the one letter codes for amino acids, terms like A21A, B28K and B29P designates that the amino acid in the A21 position is A, and the amino acids in positions 28 and 29 are lysine and proline, respectively.

Thus, e.g., B28K,B29P human insulin is an analogue of human insulin where the amino acid in position 28 in the B chain is substituted with lysine, the amino acid in position 29 in the B chain is substituted with proline, and the A chain is A(1-21).

By “desB30” or “B(1-29)” is meant a natural insulin B chain, B(1-30) which lacks the B30 amino acid and “A(1-21)” means the natural insulin A chain.

Herein the terms “A(0)” or “B(0)” indicate the positions of the amino acids N-terminally to A1 or B1, respectively. The terms A(−1) or B(−1) indicate the positions of the first amino acids N-terminally to A(0) or B(0), respectively. Thus A(−2) and B(−2) indicate positions of the amino acids N-terminally to A(−1) and B(−1), respectively, A(−3) and B(−3) indicate positions of the amino acids N-terminally to A(−2) and B(−2), respectively, and so forth. The terms A22 or B31 indicate the positions of the amino acids C-terminally to A21 or B30, respectively. The terms A23 or B32 indicate the positions of the first amino acids C-terminally to A22 or B31, respectively. Thus A24 and B33 indicate positions of the amino acids C-terminally to A23 and B32, respectively, and so forth.

In one embodiment of the invention the insulin precursor is a human insulin precursor, i.e. A is A(1-21) and B is B(1-30) in the sequence Z-B-X-Y-A. In another embodiment the insulin precursor is a desB30 human insulin precursor, i.e. A is A(1-21) and B is B(1-29). In yet another embodiment of the invention the insulin precursor has a structure wherein A and B are selected such that said insulin precursor is a precursor for B28D human insulin (aspart), B28K,B29P human insulin (lispro), B3K,B29E human insulin (glulisine), or A21G,B31R,B32R human insulin (glargine).

Herein, the term “amino acid residue” is an amino acid from which, formally, a hydroxy group has been removed from a carboxy group and/or from which, formally, a hydrogen atom has been removed from an amino group. Within the present text amino acid residue are referred to according to either their three-letter abbreviation or their one-letter abbreviation according to IUPAC nomenclature. For example, Gly and G both designate the amino acid residue glycine, and Lys and K both designate the amino acid residue lysine.

Examples of insulin analogues are such wherein Pro in position 28 of the B chain is substituted with Asp, Lys, Leu, Val, or Ala and/or Lys at position B29 is substituted with Pro, Glu or Asp. Furthermore, Asn at position B3 may be substituted with Thr, Lys, Gln, Glu or Asp. The amino acid residue in position A21 may be substituted with Gly. Also one or more amino acids may be added to the C-terminal of the A-chain and/or B-chain such as, e.g., Lys. The amino acid in position B1 may be substituted with Glu. The amino acid in position B16 may be substituted with Glu or His. Further examples of insulin analogues are the deletion analogues, e.g., analogues where the B30 amino acid in human insulin has been deleted (desB30 human insulin), insulin analogues wherein the B1 amino acid in human insulin has been deleted (desB1 human insulin), desB28-B30 human insulin and desB27 human insulin. Insulin analogues wherein the A-chain and/or the B-chain have an N-terminal extension and insulin analogues wherein the A-chain and/or the B-chain have a C-terminal extension such as with two arginine residues added to the C-terminal of the B-chain are also examples of insulin analogues. Further examples are insulin analogues comprising combinations of the mentioned mutations. Insulin analogues wherein the amino acid in position A14 is Asn, Gln, Glu, Arg, Asp, Gly or His, the amino acid in position B25 is His and which optionally further comprises one or more additional mutations are further examples of insulin analogues. Insulin analogues of human insulin wherein the amino acid residue in position A21 is Gly and wherein the insulin analogue is further extended in the C-terminal with two arginine residues are also examples of insulin analogues.

By “insulin derivative” as used herein is intended to mean a naturally occurring insulin or an insulin analogue which has been chemically modified, e.g. by introducing a side chain in one or more positions of the insulin backbone or by oxidizing or by reducing groups of the amino acid residues in the insulin or by acylating a free amino group or hydroxyl group. Non-limiting examples of insulin derivatives are e.g. N^{εB29}-tetradecanoyl des(B30) human insulin, N^{εB29}-lithocholoyl-γ-glutamyl des(B30) human insulin, N^{εB29}-(N^{α}-(HOOC(CH₂)₁₄CO)-γ-Glu) des(B30) human insulin and N^{εB29}-(N^{α}-(HOOC(CH₂)₁₆CO)-γ-Glu) des(B30) human insulin.

“Insulin precursor” as used herein is intended to mean a single-chain polypeptide which by one or more subsequent chemical and/or enzymatic processes can be converted into human insulin or an analogue thereof.

By “connecting peptide” or “C-peptide” is meant a connection moiety “C” of the B-C-A polypeptide sequence of a single chain proinsulin-molecule. In the human insulin chain, the C-peptide connects position 30 of the B chain and position 1 of the A chain and is 35 amino acid residue long. Non-limiting examples of smaller C-peptides are e.g. AAK, AAR and DKAAK.

“Mature human insulin or an analogue thereof” as used herein is intended to mean a two-chain insulin having insulin activity and with the correct amino acid residue composition and the same structural conformation as the natural insulin molecule, i.e. with disulphide bridges between positions A7-B7, A20-B19 and A6-A11. Hence, a precursor of human insulin or an analogue thereof which comprises a C-peptide would at least have the C-peptide excised to qualify as a mature human insulin or an analogue thereof. Non-limiting examples of mature human insulin or an analogue thereof are human insulin, DesB30 human insulin and B3K,B29E human insulin.

The present invention features novel C-peptides connecting the C-terminal of the B-chain with the N-terminal of the A-chain which increases the yield by expression in a fungi cell. The increased yield is assessed by the concentration of insulin precursor present in the spent culture supernatant relative to the concentration of insulin precursor in the spent culture supernatant from a fermentation using known C-peptides.

In one embodiment of the invention, X in the sequence Z-B-X-Y-A is selected from EA, AE, AD, DA, and AP.

In another embodiment of the invention X is X₁M where X₁ is an amino acid sequence comprising from 1 to 3 amino acid residues. X₁ may consist of one amino acid residue, two amino acid residues or three amino acid residues. Preferably, all the amino acid residues in X₁ are selected from the amino acid residues having side chains which are straight or branched aliphatic and side chains having a hydroxy-, carboxylic- or amide-group. In one embodiment X₁ is selected from D, SDD, and A. In another embodiment X₁ is selected from D, SDD, A, T, GD, TD, SD, ADD, DDA, N, S, GN, TS, DD, GT, GA, AD, GS, Q, ND, STD, DA, TN, SGD, TT, M, L, R, V, GDD, DTD, ST, I, TA, DGD, K, H, SS, TGD, E, TDD, G, AGD, AA, SA and AS. In another embodiment X₁ is selected from D, SDD, A, T, GD, TD, SD, ADD, DDA and N. In yet another embodiment X₁ is selected from S, GN, TS, DD, GT, GA, AD, GS, Q, ND, STD, DA, TN, SGD, TT, M, L, R and V. In another embodiment X₁ is selected from GDD, DTD, ST, I, TA, DGD, K, H, SS, TGD, E, TDD, G, AGD, AA, SA and AS. In yet another embodiment X₁ comprises no amino acid residue being P. In yet another embodiment X₁ comprises no amino acid residue being C. In yet another embodiment X₁ comprises no amino acid residue being selected from H, Y, W and F. In yet another embodiment X₁ comprises no amino acid residue being selected from K and R. In yet another embodiment X₁ comprises no amino acid residue being selected from P, C, K, R, H, Y, W, and F. Thus, in one embodiment all of the amino acids present in X₁ are selected from G, A, V, L, I, M, Q, N, E, D, S and T.

In another embodiment X is selected from EA, AE, AD, DA, and AP.

In another embodiment Y in the sequence Z-B-X-Y-A is K. The expression in a fungi cell also allows for Y to be R.

In a further embodiment X-Y in the sequence Z-B-X-Y-A is selected from SDDMK, DMK and AMK. In another embodiment X-Y in the sequence Z-B-X-Y-A is selected from SDDMK, SDMK, DMK and AMK. In yet another embodiment X-Y in the sequence Z-B-X-Y-A is selected from SDDMR, SDMR, DMR and AMR.

In a further embodiment X-Y in the sequence Z-B-X-Y-A is DMK and A and B are selected such that said insulin precursor is a precursor for B28D human insulin (aspart), i.e. A is A(1-21) and B is 28D-B(1-29). In a further embodiment X-Y in the sequence Z-B-X-Y-A is AMK and A and B are selected such that said insulin precursor is a precursor for B28D human insulin (aspart), i.e. A is A(1-21) and B is 28D-B(1-29).

The insulin precursors of the present invention may comprise an optional extension sequence, Z, in the sequence Z-B-X-Y-A. In one embodiment Z is absent, i.e. the insulin precursor has the sequence B-X-Y-A. In another embodiment Z has the sequence Z₁PK wherein Z₁ is a sequence having from 0 to 10 amino acid residues. In one embodiment Z is EEGEPK. In another embodiment Z is selected from EEAEPK, EEAEAEPK, EEAEAPK and EEAEAEAPK.

“POT” as used herein is intended to mean the Schizosaccharomyces pombe triose phosphate isomerase gene. “TPI1” as used herein is intended to mean the Saccharomyces cerevisiae triose phosphate isomerase gene.

“Leader sequence” as used herein is intended to mean an amino acid sequence consisting of a pre-peptide (the signal peptide) and a pro-peptide. Non-limiting examples of leader sequences are e.g. the alpha-factor signal leader from S. cerevisiae and the synthetic leader sequences for yeast described in WO95/34666.

“Pre-peptide” as used herein is intended to mean a signal peptide which is present as an N-terminal sequence on the precursor form of a protein. The function of the signal peptide is to allow the heterologous protein to facilitate translocation into the endoplasmic reticulum. The signal peptide is normally cleaved off in the course of this process. The signal peptide may be heterologous or homologous to the fungi organism producing the protein. A number of signal peptides which may be used with the DNA construct of the invention including yeast aspartic protease 3 (YAP3) signal peptide or any functional analog (Egel-Mitani et al. (1990) YEAST 6:127-137 and U.S. Pat. No. 5,726,038) and the a-factor signal of the MFα1 gene, Thorner (1981) in The Molecular Biology of the Yeast Saccharomyces cerevisiae, Strathern et al., eds., pp 143-180, Cold Spring Harbor Laboratory, NY and U.S. Pat. No. 4,870,00.

“Pro-peptide” as used herein is intended to mean a polypeptide sequence whose function is to allow the expressed polypeptide to be directed from the endoplasmic reticulum to the Golgi apparatus and further to a secretory vesicle for secretion into the culture medium (Le. exportation of the polypeptide across the cell wall or at least through the cellular membrane into the periplasmic space of the yeast cell). The pro-peptide may be the yeast a-factor pro-peptide, vide U.S. Pat. Nos. 4,546,082 and 4,870,008. Alternatively, the pro-peptide may be a synthetic pro-peptide, which is to say a pro-peptide not found in nature. Suitable synthetic pro-peptides are those disclosed in U.S. Pat. Nos. 5,395,922; 5,795,746; 5,162,498 and WO 98/32867. The pro-peptide will preferably contain an endopeptidase processing site at the C-terminal end, such as a Lys-Arg sequence or any functional analog thereof.

The polynucleotide sequence of the invention may be prepared synthetically by established standard methods, e.g. the phosphoamidite method described by Beaucage et al. (1981) Tetrahedron Letters 22:1859-1869, or the method described by Matthes et al. (1984) EMBO Journal 3:801-805. According to the phosphoamidite method, oligonucleotides are synthesized, for example, in an automatic DNA synthesizer, purified, duplexed and ligated to form the synthetic DNA construct. One way of preparing the DNA construct is by polymerase chain reaction (PCR).

The polynucleotide sequence of the invention may also be of mixed genomic, cDNA, and synthetic origin. For example, a genomic or cDNA sequence encoding a leader peptide may be joined to a genomic or cDNA sequence encoding the A and B chains, after which the DNA sequence may be modified at a site by inserting synthetic oligonucleotides encoding the desired amino acid sequence for homologous recombination in accordance with well-known procedures or preferably generating the desired sequence by PCR using suitable oligonucleotides.

The invention encompasses a vector which is capable of replicating in the selected microorganism or host cell and which carries a polynucleotide sequence encoding the insulin precursors or insulin precursor analogues of the invention. The recombinant vector may be an autonomously replicating vector, i.e., a vector which exists as an extra-chromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The vector may be linear or closed circular plasmids and will preferably contain an element(s) that permits stable integration of the vector into the host cell's genome or autonomous replication of the vector in the cell independent of the genome.

In a preferred embodiment, the recombinant expression vector is capable of replicating in yeast Examples of sequences which enable the vector to replicate in yeast are the yeast plasmid 2μ replication genes REP 1-3 and origin of replication.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol or tetracycline resistance.

Selectable markers for use in a filamentous fungal host cell include amdS (acetamidase), argB (ornithine carbamoyltransferase), pyrG (orotidine-5′-phosphate decarboxylase) and trpC (anthranilate synthase). Examples of suitable markers for yeast host cells are ADE2, HIS3, LEU2, LYS2, MET3, TRP1, and URA3. A preferred selectable marker for yeast is the Schizosaccharomyces pombe TPI gene (Russell (1985) Gene 40:125-130).

In the vector, the polynucleotide sequence is operably connected to a suitable promoter sequence. The promoter may be any nucleic acid sequence which shows transcriptional activity in the host cell of choice including mutant, truncated, and hybrid promoters, and may be obtained from genes encoding extra-cellular or intra-cellular polypeptides either homologous or heterologous to the host cell.

Examples of suitable promoters for directing the transcription in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), and Bacillus licheniformis penicillinase gene (penP). Examples of suitable promoters for directing the transcription in a filamentous fungal host cell are promoters obtained from the genes for Aspergillus oryzae TAKA amylase, Rhizomucor miehei aspartic proteinase, Aspergillus niger neutral alpha-amylase, and Aspergillus niger acid stable alpha-amylase. In a yeast host, examples of useful promoters are the Saccharomyces cerevisiae MFα1, TPI1, ADH, TDH3 or PGK promoters.

The polynucleotide construct of the invention will also typically be operably connected to a suitable terminator. In yeast a suitable terminator is the TPI1 terminator (Alber et al. (1982) J. Mol. Appl. Genet. 1:419-434).

The procedures used to ligate the polynucleotide sequence of the invention, the promoter and the terminator, respectively, and to insert them into suitable yeast vectors containing the information necessary for yeast replication, are well known to persons skilled in the art. It will be understood that the vector may be constructed either by first preparing a DNA construct containing the entire DNA sequence encoding the insulin precursors or insulin precursor analogues of the invention, and subsequently inserting this fragment into a suitable expression vector, or by sequentially inserting DNA fragments containing genetic information for the individual elements (such as the signal, pro-peptide, mini C-peptide, A and B chains) followed by ligation.

The present invention also relates to recombinant host cells, comprising a polynucleotide sequence encoding the insulin precursors or the insulin precursor analogues of the invention. A vector comprising such polynucleotide sequence is introduced into the host cell so that the vector is maintained as a chromosomal integrant or as a self-replicating extrachromosomal vector.

“Host cell” as used herein is intended to mean a microorganism which is used for the expression of a polypeptide of interest. A host cell encompasses any progeny of a parent cell that is not identical to the parent cell due to mutations that occur during replication.

Suitable host cells for the present invention is a fungal cell. “Fungi” as used herein is intended to include the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In one embodiment the host cell is a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Pichia, Kluyveromyces and Saccharomyces). The basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (e.g., genera Sorobolomyces and Bullera) and Cryptococcaceae (e.g., genus Candida). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathern et al., editors, 1981).

The yeast host cell used in the process of the invention may be any suitable yeast organism which, on cultivation, produces large amounts of the insulin precursor and insulin precursor analogues of the invention.

Examples of suitable yeast organisms are strains selected from a cell of a species of Candida, Kluyveromyces, Saccharomyces, Schizosaccharomyces, Pichia, Hansenula, and Yarrowia. In one embodiment, the yeast host cell is selected from a Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Schizosaccharomyces pombe, Sacchoromyces uvarum, Pichia kluyveri, Yarrowia lipolytica, Candida utilis, Candida cacaoi, and Geotrichum fermentans. Other useful yeast host cells are a Kluyveromyces lactis, Kluyveromyces fragilis, Hansenula polymorpha, Pichia pastoris Yarrowia lipolytica, Schizosaccharomyces pombe, Ustilgo maylis, Candida maltose, Pichia guillermondii and Pichia methanoliol (cf. Gleeson et al., J. Gen. Microbiol. 132, 1986, pp. 3459-3465; U.S. Pat. No. 4,882,279 and U.S. Pat. No. 4,879,231). The transformation of the yeast cells may for instance be effected by protoplast formation followed by transformation in a manner known per se.

In one embodiment the host cell is a filamentous fungal cell. “Filamentous fungi” include all filamentous forms of the subdivision Eumycota and Oomycota (as defined by Hawksworth et al., 1995, supra). The filamentous fungal host cell may be chosen from the group consisting of Acremonium, Aspergillus, Fusarium, Humicola, Mucor, Myceliophthora, Neurospora, Penicillium, Thielavia, Tolypocladium, and Trichoderma.

The host cell for expressing the insulin precursors is preferably a cell free from any functional antibiotic resistance genes. Although such antibiotic resistance genes are useful during initial cloning steps in e.g. E. coli, the antibiotic resistance genes can be made non-functional or removed from the host cell by well known procedures, see e.g. WO 00/04172.

“Medium” as used herein is intended to mean a liquid solution for cultivating the host cell, i.e. supporting the growth and product formation of the fungi. A suitable medium for fungi is e.g. YPD or as described in WO2008/037735. The medium contains at least one carbon source, one or several nitrogen sources, essential salts including salts of potassium, sodium, magnesium, phosphate and sulphate, trace metals, water soluble vitamins, and process aids including but not limited to antifoam agents, protease inhibitors, stabilizers, ligands and inducers. Typical carbon sources are e.g. mono- or disaccharides. Typical nitrogen sources are, e.g. ammonia, urea, amino acids, yeast extract, corn steep liquor and fully or partially hydrolysed proteins. Typical trace metals are e.g. Fe, Zn, Mn, Cu, Mo and H₃BO₃. Typical water soluble vitamins are e.g. biotin, pantothenate, niacin, thiamine, p-aminobenzoic acid, choline, pyridoxine, folic acid, riboflavin and ascorbic acid.

By “fermentation” as used herein is intended to mean an aseptic process used for propagating microorganisms submerged in a liquid medium. The fermentation is preferably carried out in aseptic, stirred tanks with supply lines for addition of compressed, sterile gasses consisting of but not limited to air, oxygen and ammonia. A fermentation tank can contain sensors and devices for monitoring pH, temperature, pressure, agitation rate, dissolved oxygen level, liquid content, foam level, feed addition rates and rates of adding acid and base. Furthermore, the fermentation tank can be equipped with optical devises for monitoring levels of cell density, concentrations of metabolites and products regardless of their physio-chemical form.

The desired product produced during the fermentation is present as soluble extracellular material or as intracellular material either in the form of soluble material or as insoluble material including aggregated material. A fermentation process is typically carried out in tanks with a working volume ranging from 100 mL to 200.000 L. A fermentation process can be operated as a batch process, a fed-batch process, a repeated fed-batch process or a continuous process.

The secreted insulin precursor or insulin analogue precursor, a significant proportion of which will be present in the medium in correctly processed form, may be recovered from the medium by conventional procedures including separating the yeast cells from the medium by centrifugation, filtration or catching the precursor of human insulin or analogue thereof by an ion-exchange matrix or by a reverse phase absorption matrix, precipitating the proteinaceous components of the supernatant or filtrate by means of a salt, e.g. ammonium sulphate, followed by purification by a variety of chromatographic procedures, e.g. ion exchange chromatography, affinity chromatography, or the like.

The precursor of human insulin or analogue thereof of the invention may be expressed with an N-terminal amino acid residue extension, as described in U.S. Pat. No. 5,395,922 and European Patent No. 765,395A. The extension is found to be stably attached to the precursor of human insulin or analogue thereof of the invention during fermentation, protecting the N-terminal end of the insulin precursor or insulin precursor analog against the proteolytic activity of yeast proteases such as DPAP. The presence of an N-terminal extension on the precursor of human insulin or analogue thereof may also serve as a protection of the N-terminal amino group during chemical processing of the protein, i.e. it may serve as a substitute for a BOC (t-butyl-oxycarbonyl) or similar protecting group. The N-terminal extension may be removed from the recovered insulin precursor or insulin precursor analog by means of a proteolytic enzyme which is specific for a basic amino acid (e.g., Lys) so that the terminal extension is cleaved off at the Lys residue. Examples of such proteolytic enzymes are trypsin and Achromobacter lyticus protease.

After secretion to the culture medium and recovery, the insulin precursor or insulin precursor analogues of the invention will be subjected to various in vitro procedures to remove

the possible N-terminal extension sequence and the C-peptide to give insulin or the desired insulin analogue. Such methods include enzymatic conversion by means of trypsin or an Achromobacter lyticus protease in the presence of an L-threonine ester followed by conversion of the threonine ester of the insulin or insulin analogue into insulin or the insulin analogue by basic or acid hydrolysis as described in U.S. Pat. Nos. 4,343,898 or 4,916,212 or Research Disclosure, September 1994/487 the disclosures of which are incorporated by reference hereinto.

As described below, insulin precursors or insulin precursor analogues with synthetic C-peptides were constructed (Example 1).

Saccharomyces cerevisiae expression plasmids containing a polynucleotide sequence encoding the claimed insulin precursors or insulin analogue precursor were constructed by PCR and used to transform a S. cerevisiae host cell. The amount of expressed product, e.g. an insulin analogue was measured as a percentage of the relevant control level from expression in yAK1220, i.e. the precursor EEAEAEAPK-(B(1-29)-AAK-A(1-21) with the alpha leader. The novel C-peptides of the invention gave increased yields by up to 300% and they caused general reductions in the O-glycosylation levels. Also the insulin precursors comprising the novel C-peptides exhibit good excision of the C-peptide by e.g. Acromobacter lyticus protease.

The cleavage efficiency of Acromobacter lyticus protease when used to cleave an insulin precursor according to the present invention may be determined by a simple assay as follows: A suitable aqueous solution of the insulin precursor is incubated at a pH and temperature which is favourable to A. lyticus protease and samples are withdrawn from the reaction mixture over time. As soon as the samples are withdrawn the enzyme activity is inactivated. After collecting all the samples covering the time-span of interest, the concentration of the corresponding mature insulin polypeptide is determined by e.g. HPLC analysis. Depicting the concentration of the mature insulin polypeptide as a function of time will indicate the progress of the reaction. Comparing such reaction traces for different insulin precursors cleaved under identical reaction conditions will allow a ranking of the insulin precursors in accordance with ability to be matured by the action of A. lyticus protease. A similar procedure can be applied for other proteases which may be chosen for conversion of the insulin precursor into the corresponding mature insulin polypeptide.

The novel C-peptides of the present invention also exhibit reduced O-glycosylation of the precursor for human insulin or an analogue thereof during expression in a fungi cell. As such the precursors of human insulin or an analogue thereof according to the present invention can be used in an improved method for making mature human insulin or an analogue thereof in a fungi cell. Expressing the precursors for human insulin or an analogue thereof according to the invention in a fungi cell having reduced capacity for O-glycosylation may maintain the improved yield of said precursor while at the same time reducing even further the fraction of said precursor molecule that is O-glycosylated during expression.

Protein O-mannosyltransferases (PMTs) initiate the assembly of O-mannosyl glycans, an essential protein modification in fungi. PMTs are conserved in fungi and the PMT family is phylogenetically classified into PMT1, PMT2 and PMT4 subfamilies, which differ in protein substrate specificity. The protein O-mannosyltransferases Pmt1p and Pmt2p are catalyzing the O-glycosylation of serine and threonine residues in proteins in the endoplasmic reticulum of yeast by transfer of a mannosyl residue from Dolichyl phosphate-D-mannose (Gentzsch et al., FEBS Lett 1995, 18, pp 128-130). In Saccharomyces cerevisiae as well as in many other fungi the PMT family is highly redundant, and only the simultaneous deletion of PMT1/PMT2 and PMT4 subfamily members is lethal (Girrbach and Strahl, J. Biol. Chem. 2003, 278, pp 12554-62). U.S. Pat. No. 5,714,377 describe that fungal cells having reduced O-glycosylation capacity from PMT1/PMT2 modification are still viable and show good growth characteristics in industrial fermentation conditions.

The invention is further described by the following non-limiting embodiments:
1. Insulin precursor comprising the sequence Z-B-X-Y-A wherein
   Z is an optional extension sequence,
   B is the B-chain of human insulin or an analogue thereof,
   X is a sequence selected from X₁M, EA, AE, AD, DA, AP, AW, and LA, where X₁ is a sequence comprising from 1 to 3 amino acid residues,
   Y is K or R, and
   A is the A-chain of human insulin or an analogue thereof.
2. The insulin precursor according to embodiment 1 wherein X is a sequence selected from X₁M, EA, AE, AD, DA and AP, where X₁ is a sequence comprising from 1 to 3 amino acid residues.
3. The insulin precursor according to any of embodiments 1-2 wherein X is X₁M.
4. The insulin precursor according to embodiment 3 wherein X₁ is selected from D, SDD, A, T, GD, TD, SD, ADD, DDA, N, S, GN, TS, DD, GT, GA, AD, GS, Q, ND, STD, DA, TN, SGD, TT, M, L, R, V, GDD, DTD, ST, I, TA, DGD, K, H, SS, TGD, E, TDD, G, AGD, AA, SA and AS.
5. The insulin precursor according to any of embodiments 1-4 wherein X₁ is selected from D, SDD, A, T, GD, TD, SD, ADD, DDA and N.
6. The insulin precursor according to embodiment 5 wherein X₁ is selected from D, SDD and A.
7. The insulin precursor according to any of embodiments 1-4 wherein X₁ is selected from S, GN, TS, DD, GT, GA, AD, GS, Q, ND, STD, DA, TN, SGD, TT, M, L, R and V.
8. The insulin precursor according to any of embodiments 1-4 wherein X₁ is selected from GDD, DTD, ST, I, TA, DGD, K, H, SS, TGD, E, TDD, G, AGD, AA, SA and AS.
9. The insulin precursor according to any of embodiments 1-8, wherein all the amino acid residues in X₁ are selected from the amino acid residues having side chains which are straight or branched aliphatic and side chains having a hydroxy-, carboxylic- or amide-group.
10. The insulin precursor according to any of embodiments 1-9 wherein X₁ comprises no amino acid residue being P.
11. The insulin precursor according to any of embodiments 1-10 wherein X₁ comprises no amino acid residue being C.
12. The insulin precursor according to any of embodiments 1-11 wherein X₁ comprises no amino acid residue being selected from H, Y, W and F.
13. The insulin precursor according to any of embodiments 1-12 wherein X₁ comprises no amino acid residue being selected from K and R.
14. The insulin precursor according to any of embodiments 1-13 wherein X₁ comprises no amino acid residue being selected from P, C, K, R, H, Y, W and F.
15. The insulin precursor according to any of embodiments 1-14 wherein all of the amino acid residues in X₁ are selected from G, A, V, L, I, M, Q, N, E, D, S and T.
16. The insulin precursor according to any of embodiments 1-2 wherein X is selected from EA, AE, AD, DA, AP, AW, and LA.
17. The insulin precursor according to embodiment 16 wherein X is selected from EA, AE, AD, DA and AP.
18. The insulin precursor according to any of embodiments 1-15 wherein X₁ consists of one amino acid residue.
19. The insulin precursor according to any of embodiments 1-15 wherein X₁ consists of two amino acid residues.
20. The insulin precursor according to any of embodiments 1-15 wherein X₁ consists of three amino acid residues.
21. The insulin precursor according to any of embodiments 1-20 wherein Y is K.
22. The insulin precursor according to any of embodiments 1-20 wherein Y is R.
23. The insulin precursor according to any of embodiments 1-21 wherein X-Y is selected from AMK, DMK, SDDMK and SDMK.
24. The insulin precursor according to any of embodiments 1-20 wherein X-Y is selected from AMR, DMR, SDDMR and SDMR.
25. The insulin precursor according to any of embodiments 1-24 which is a human insulin precursor, i.e. A is A(1-21) and B is B(1-30).
26. The insulin precursor according to any of embodiments 1-24 which is a desB30 human insulin precursor, i.e. A is A(1-21) and B is B(1-29).
27. The insulin precursor according to any of embodiments 1-24, wherein A and B are selected such that said insulin precursor is B28D human insulin (aspart), B28K,B29P human insulin (lispro), B3K,B29E human insulin (glulisine), or A21G,B31R,B32R human insulin (glargine).
28. The insulin precursor according to any of embodiments 1-27 wherein Z is absent.
29. The insulin precursor according to any of embodiments 1-27, wherein Z is a peptide consisting of from about 3 to about 20 amino acid residues.
30. The insulin precursor according to embodiment 29, wherein Z is a peptide consisting of from about 5 to about 15 amino acid residues.
31. The insulin precursor according to any of embodiments 1-27 and 29-30, wherein the C-terminal of Z is EPK or APK.
32. The insulin precursor according to any of embodiments 1-27 and 29-30, wherein Z has the sequence Z₁PK wherein Z₁ is a sequence having from 0 to 10 amino acid residues.
33. The insulin precursor according to embodiment 32, wherein Z₁ comprises at least two amino acid residues being E.
34. The insulin precursor according to any of embodiments 32-33, wherein Z₁ comprises at least two amino acid residues being A.
35. The insulin precursor according to any one of embodiments 32-34, wherein Z₁ comprises at least one subsequence being EA.
36. The insulin precursor according to embodiment 29 wherein Z is EEGEPK.
37. The insulin precursor according to embodiment 29 wherein Z is selected from EEAEPK, EEAEAEPK, EEAEAPK and EEAEAEAPK.
38. Method for making mature human insulin or an analogue thereof said method comprising (i) culturing a fungi cell comprising a DNA sequence encoding a precursor for human insulin or an analogue thereof according to any of embodiments 1-37 under suitable culture conditions for expression of said precursor for human insulin or an analogue of human insulin; and (ii) isolating the expressed precursor.
39. Method for reducing O-glycosylation of a precursor for human insulin or an analogue of human insulin during expression in a fungi cell, said method comprising (i) culturing a fungi cell comprising a DNA sequence encoding a precursor for human insulin or an analogue thereof according to any of embodiments 1-37 under suitable culture conditions for expression of said precursor for human insulin or an analogue of human insulin.
40. Method for increasing the yield of a precursor for human insulin or an analogue of human insulin during expression in a fungi cell, said method comprising (i) culturing a fungi cell comprising a DNA sequence encoding a precursor for human insulin or an analogue thereof insulin according to any of embodiments 1-37 under suitable culture conditions for expression of said precursor for human insulin or an analogue of human insulin.
41. The method according to any of embodiments 38-40 wherein said fungi cell carries at least one genetic modification reducing its capacity for O-glycosylation.
42. The method according to embodiment 41 wherein said fungi cell carries at least one genetic modification reducing its capacity for O-glycosylation of the des-B30 human insulin precursor EEAEAEAPK-B(1-29)-AAK-A(1-21) when expressed with the alpha leader by protein O-mannosyltransferase 1 (PMT1) as compared to the fungi cell carrying the corresponding unmodified genes.
43. The method according to embodiment 41 wherein said fungi cell carries at least one genetic modification reducing its capacity for O-glycosylation of the des-B30 human insulin precursor EEAEAEAPK-B(1-29)-AAK-A(1-21) when expressed with the alpha leader by protein O-mannosyltransferase 2 (PMT2) as compared to the fungi cell carrying the corresponding unmodified genes.
44. The method according to any of embodiments 41-43 wherein said capacity for 0-glycosylation is reduced by at least a factor 2.
45. The method according to any of embodiments 41-44 wherein said capacity for 0-glycosylation is reduced by at least a factor 4.
46. The method according to any of embodiments 41-45 wherein said at least one genetic modification is located in the coding region of PMT1 or PMT2.
47. The method according to any of embodiments 41-45 wherein said fungi cell carries at least one genetic modification within the genes for PMT1 or PMT2 reducing its capacity for O-glycosylation.
48. The method according to any of embodiments 41-45 wherein said at least one genetic modification is located in the regions responsible for or involved in the expression and/or transcriptional regulation of PMT1 or PMT2.
49. The method according to any of embodiments 41-48 wherein the PMT1 and PMT2 genes in said fungi cell are both deleted.
50. The method according to any of embodiments 38-49 wherein said DNA sequence encoding a precursor of human insulin or an analogue of human insulin comprises a leader sequence.
51. The method according to embodiment 50 wherein said leader sequence is selected from the group consisting of alpha-factor signal leader, alpha2, alpha4, LA19 and TA39.
52. The method according to any of embodiments 38-51 wherein said fungi cell is a yeast.
53. The method according to embodiment 52 wherein said yeast is Saccharomyces cerevisiae.
54. The method according to embodiment 52 wherein said yeast is Pichia pastoris.
55. The method according to embodiment 52 wherein said yeast is Hansenula polymorpha.
56. Polynucleotide sequence encoding a precursor for human insulin or an analogue of human insulin according to any of embodiments 1-37.
57. Expression vector comprising a polynucleotide sequence according to embodiment 56.
58. Host cell transformed with a vector according to embodiment 57.

The present invention is described in further detain in the following examples which are not in any way intended to limit the scope of the invention as claimed. The attached Figures are meant to be considered as integral parts of the specification and description of the invention. All references cited are herein specifically incorporated by reference for all that is described therein.

### EXAMPLES

### General Procedures

### Expression Plasmids

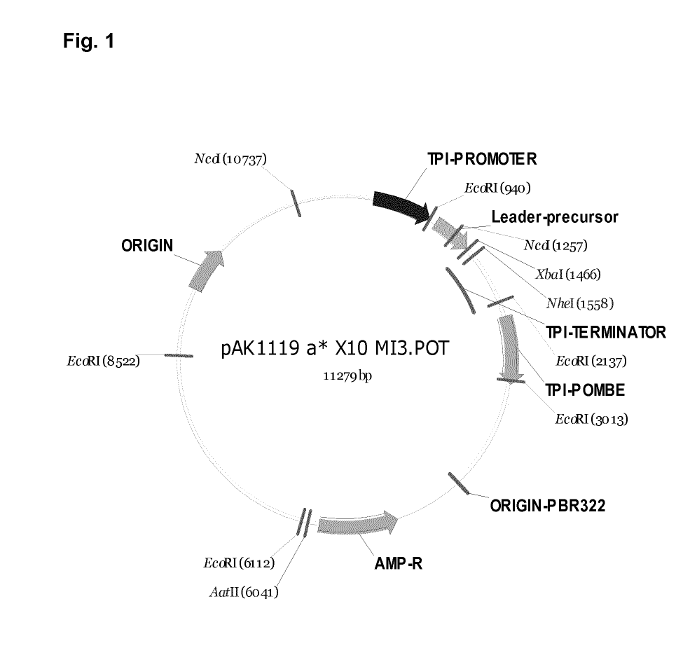

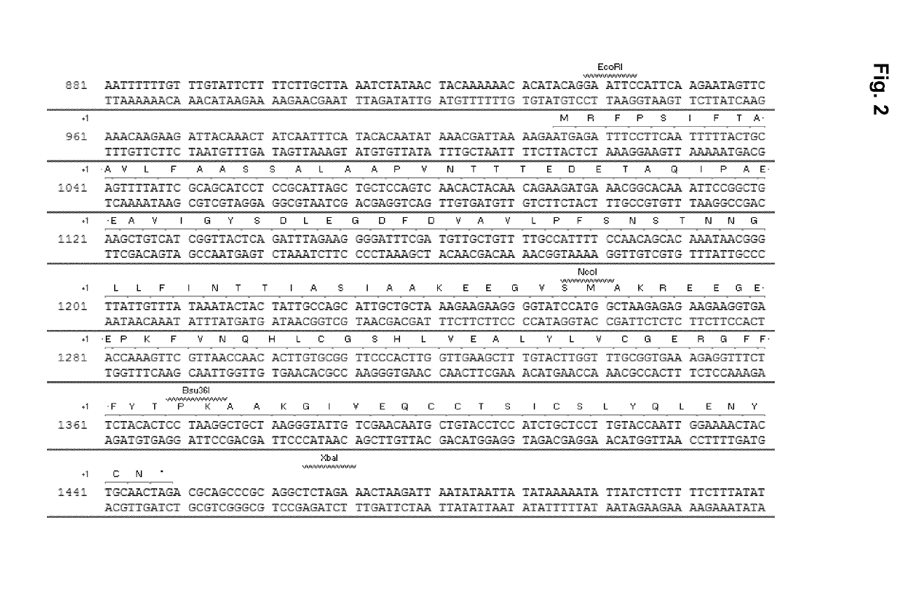

All expressions plasmids are of the C-POT type, similar to those described in EP 171,142. These are 2μ-based expression vectors characterized by containing the Schizosaccharomyces pombe triose phosphate isomerase gene (POT) for the purpose of plasmid selection and stabilization in S. cerevisiae. The plasmids also contain the S. cerevisiae triose phosphate isomerase promoter and terminator (FIG. 1). These sequences are similar to the corresponding sequences in plasmid pKFN1003 (described in WO 90/10075) as are all sequences except the following: 1) the sequence of the EcoRI-XbaI fragment encoding the fusion protein of the leader and the insulin product and 2) a silent mutation has been introduced resulting in removal of a NcoI-site in the 2μ-region in the expression vector. In order to facilitate cloning of different fusion proteins the DNA sequence encoding the MFα1 pre-pro leader has been changed to incorporate a NcoI site (see FIG. 2) and is called the MFα1* pre-pro leader. Thus the NcoI-XbaI fragment is simply replaced by an NcoI-XbaI fragment encoding the insulin construct of interest. Such NcoI-XbaI fragments may be synthesized using synthetic oligonucleotides and PCR according to standard techniques. In addition to the alpha-leader other leaders can be used, as described in the Examples below.

### Yeast Transformation

Yeast transformants were prepared by transformation of the host strains S. cerevisiae strain MT663. The yeast strain MT663 (MATa/MATα pep4-3/pep4-3 HIS4/his4 Δtpi1::LEU2/Δtpi1::LEU2 Cir′) was deposited in the Deutsche Sammlung von Mikroorganismen and Zellkulturen in connection with filing WO 92/11378 and was given the deposit number DSM 6278.

MT663 is grown on YPGGE (1% Bacto yeast extract, 2% Bacto peptone, 2% galactose, 1% EtOH, 2% glycerol) to an O.D. at 600 nm of 0.2. 100 ml of culture was harvested by centrifugation, washed with 10 ml of water, recentrifuged and resuspended in 10 ml of a solution containing 1 M sorbitol, 25 mM Na₂EDTA pH=8.0 and 6.7 mg/ml dithiotreitol. The suspension was incubated at 30° C. for 15 minutes, centrifuged and the cells resuspended in 10 ml of a solution containing 1.2 M sorbitol, 10 mM Na₂EDTA. 0.1 M sodium citrate, pH 0 5.8, and 2 mg NovozymC3234. The suspension was incubated at 30° C. for 30 minutes, the cells collected by centrifugation, washed in 10 ml of 1.2 M sorbitol and 10 ml of CAS (1.2 M sorbitol, 10 mM CaCl₂, 10 mM Tris HCl (Tris=Tris(hydroxymethyl)-aminomethane) pH=7.5) and resuspended in 2 ml of CAS. For transformation, 1 ml of CAS-suspended cells was mixed with approx. 0.1 μg of plasmid DNA and left at room temperature for 15 minutes. 1 ml of (20% polyethylene glycol 4000, 10 mM CaCl₂, 10 mM Tris HCl, pH=7.5) was added and the mixture left for a further 30 minutes at room temperature. The mixture was centrifuged and the pellet resuspended in 0.1 ml of SOS (1.2 M sorbitol, 50% YPGGE, 6.7 mM CaCl₂) and incubated at 30° C. for 2 hours. The suspension was then centrifuged and the pellet resuspended in 0.5 ml of 1.2 M sorbitol. Then, 6 ml of top agar (the SC medium of Sherman et al. (1982) Methods in Yeast Genetics, Cold Spring Harbor Laboratory) containing 2% glucose, plus 2.5% agar) at 52° C. was added and the suspension poured on top of plates containing the same agar-solidified, sorbitol containing medium.

### Yeast Fermentations

S. cerevisiae strain MT663 transformed with expression plasmids was grown in YPD medium for 72 h at 30° C.

### Quantification of Glycosylation Levels

Glycosylation levels of the insulin precursor in the culture supernatants were determined using an LC-MS system interfacing Waters Acquity UPLC system (Waters, Milford, Mass., USA) consisting of an autosampler (Model Acq-SM), pump (Model Acq-BSM), column oven (Model Acq-SM) and detector (Model Acq-TUV) with LTQ Orbitrap XL (Thermo Fisher, Waltham, Mass., USA). RP-HPLC separation was achieved using a linear gradient of acetonitrile in 0.1% formic acid (0 min 12% acetonitrile, 10 min 15% acetonitrile, 27 min 40% acetonitrile, 27.5 min 90% acetonitrile) using CSH C18 column (Waters, 1×150 mm) with a flow rate of 0.1 ml/min at 45° C. LTQ Orbitrap was tuned according to manufacturer's instructions and operated in positive mode with ESI source (Source Voltage of 4000 V, Capillary Temp of 325° C., Sheath Gas Flow of 40, Aux Gas Flow of 10 and Sweep Gas Flow of 2). Full FTMS scan (m/z=900-2000) with resolution of 30000 was used to collect data.

Yeast cultures were centrifuged (5000 rpm, 5 min) and supernatant analyzed by LC-MS either directly or after processing with A. lyticus protease. O-glycosylation levels were obtained after deconvolution as a ratio of intensity corresponding to the mass of o-glycosylated insulin species (mono-o-glycosylated species M+162 Da) and the intensity corresponding to the mass of non-glycosylated species (M) and expressed in percent. This method was applied to obtained levels of mono-o-glycosylated as well as multi-o-glycosylated products.

### Example 1

Synthetic genes encoding fusion proteins, consisting of the insulin precursor associated with a leader sequence consisting of a pre-peptide (signal peptide) and a pro-peptide, were constructed using PCR under standard conditions (Sambrook et al. (1989) Molecular Cloning, Cold Spring Harbor Laboratory Press) and E.H.F. polymerase (Boehringer Mannheim GmbH, Sandhoefer Strasse 116, Mannheim, Germany). The resulting DNA fragments were isolated and digested with endonucleases and purified using the QIAquick Gel Extraction Kit (QIAGEN, Hilden, Germany). Standard methods were used for DNA ligation and transformation of E. coli cells were performed by the CaCl₂ method (Sambrook et al. (1989) supra). Plasmids were purified from transformed E. coli cells using Manual Perfectprep Plasmid 96 Vac Kit (5 PRIME, Hamburg, Germany and Gaithersburg, USA) and epMotion 5075 VAC (automated pipetting system), Eppendorf, Hamburg, Germany). Nucleotide sequences were determined by eurofins MWG/operon (Ebersberg, Germany) with purified double-stranded plasmid DNA as template. Oligonucleotide primers for PCR were obtained from DNA technology (Arhus, Denmark).

Secretion of the insulin precursor was facilitated by the alpha-leader or the TA39 leader (Kjeldsen et al., 1999. Biotechnol. Appl. Biochem 29, 79-86), although a variety of known yeast leader sequences may be used.

As shown in FIGS. 1 and 2, the pAK1119 S. cerevisiae expression plasmid expressing the alpha*-leader (without the BgIII-site) (SEQ ID NO:1)-EEGEPK (SEQ ID NO:2)-insulin precursor fusion protein was constructed based on the S. cerevisiae-E. coli shuttle POT plasmid (U.S. Pat. No. 5,871,957). In FIG. 1 Leader-precursor indicates the fusion protein expression cassette encoding the leader-insulin precursor fusion protein; TPI-PROMOTER is the S. cerevisiae TPI1 promoter, TPI-TERMINATOR is the S. cerevisiae TPI1 terminator; TPI-POMBE indicates the S. pombe POT gene used for selection in S. cerevisiae; ORIGIN indicates a S. cerevisiae origin of replication derived from the 2 μm plasmid; AMP-R indicates the β-lactamase gene conferring resistance toward ampicillin, facilitating selection in E. coli; and ORIGIN-PBR322 indicates an E. coli origin of replication.

DNA encoding a number of fusions proteins of leader sequences and insulin precursors with different mini C-peptides was generated by PCR using appropriate oligonucleotides as primers, as described below. Standard methods were used to subclone DNA fragments encoding the leader-insulin precursor-fusion proteins into the CPOT expression vector in the following configuration: leader-Lys-Arg-spacer-insulin precursor, where Lys-Arg is a potential dibasic endoprotease processing site and spacer is an N-terminal extension. To optimize processing of the fusion protein by the S. cerevisiae Kex2 endoprotease, DNA encoding a spacer peptide (N-terminal extension), e.g. EEGEPK (SEQ ID NO:2), was inserted between the DNA encoding the leader and the insulin precursor (Kjeldsen, et al. 1999b. J. Biotechnology, 75, 195-208). However, the presence of the spacer peptide is not mandatory. The insulin precursor was secreted as a single-chain N-terminally extended insulin precursor with a mini C-peptide, connecting Lys^{B29} and Gly^{A1}. After purification of the insulin precursor and proteolytic removal of the N-terminal extension and the mini C-peptide, the amino acid Thr^{B30} can be added to Lys^{B29} by enzyme-mediated transpeptidation, to generate human insulin (Markussen, et al. (1987) in “Peptides 1986” (Theodoropoulos, D., Ed.), pp. 189-194, Walter de Gruyter & Co., Berlin).

Development of synthetic mini C-peptides was performed by randomization of one or more codon(s) encoding the amino acids in the mini C-peptide. The synthetic mini C-peptides feature typically an enzymatic processing site (Lys) at the C-terminus which allows enzymatic removal of the synthetic mini C-peptide. Randomization was performed using doped oligonucleotides which introduced codon(s) variations at one or more positions of the synthetic mini C-peptides. Typically one of the two primers (oligonucleotides) used for PCR was doped. An example of an oligonucleotides pair used for PCR generation of leader-insulin precursor with randomized synthetic mini C-peptides used to generated synthetic mini C-peptides with the general formula: Ala-Xaa-Lys (AXK) are as follows:

Primer A (introducing the BgIII-site):

(SEQ ID NO: 3)
5′-ATACAGGAATTCCATTCAAGATCTGTTCAAACAAGAAGA-3′

Primer B:

(SEQ ID NO: 4)
5′-AATCTTAGTTTCTAGACTAGTTGCAGTAGTTTTCCAATTGGTAC
AAGGAGCAGATGGAGGTACAGCATTGTTCGACAATACCCTTMNNAGC
CTTAGGAGTGTAGAA-3′
N = ACTG
M = AC

Polymerase chain reaction.

PCR was typically performed as indicated below: 5 μl Primer A (20 pmol/μl), 5 μl Primer B (20 pmol/μl), 10 μl 10×PCR buffer, 8 μl dNTP mix, 0.75 μl E.H.F. enzyme, 1 μl pAK1119 plasmid as template (approximately 0.2 μg DNA) and 70.25 μl distilled water.

Typically 12 cycles were performed, one cycle typically was 95° C. for 45 sec.; 48° C. for 1 min; 72° C. for 1.5 min. The PCR mixture was subsequently loaded onto a 2% agarose gel and electrophoresis was performed using standard techniques. The resulting DNA fragment was cut out of the agarose gel and isolated by the QIAquick Gel Extraction Kit.

FIG. 2 shows the nucleotide sequence of pAK1119 DNA expression cassette (SEQ ID NO:5) used as template for PCR and inferred amino acids of the encoded fusion protein (alpha*-leader-EEGEPK-insulin precursor of pAK1119 (SEQ ID NO:6).

The purified PCR DNA fragment was dissolved in Buffer EB (10 mM Tris HCl pH 8.5, provided in the QIAquick Gel Extraction Kit) and digested with suitable restriction endonucleases (e.g. Bgl II and Xba I) according to standard techniques. The BgIII-XbaI DNA fragments were subjected to agarose electrophoresis and purified using the QIAquick Gel Extraction Kit.

The digested and isolated DNA fragments were ligated together with a suitable vector (e.g. of the CPOT type) using T4 DNA ligase and standard conditions. The ligation mix was subsequently transformed into a competent E. coli strain followed by selection with ampicillin resistance. Plasmids from the resulting E. coli's were isolated using Manual Perfectprep Plasmid 96 Vac Kit and epMotion 5075 VAC (automated pipetting system).

The plasmids were subsequently used for transformation of a suitable S. cerevisiae host strain, e.g., MT663 (MATa/MATα pep4-3/pep4-3 HIS4/his4 tpi1::LEU2/tpi1::LEU2 Cir⁺). Individual transformed S. cerevisiae clones were grown in liquid culture, and the quantity of the insulin precursor secreted to the culture supernatants was determined by RP-HPLC. The DNA sequence encoding the synthetic mini C-peptide of the expression plasmids from S. cerevisiae clones secreting increased quantity of the insulin precursor were then determined. Subsequently, the identified synthetic mini C-peptide sequence might be subjected to another round of randomization optimization.

### Examples 2-84

Insulin precursors and expression constructs according to the present invention were generated by the method described in Example 1. Table 1 shows the insulin precursors and the corresponding production yield (expressed as a percent of the control, YAK1220) and the O-glycosylation level. Fermentations were all conducted at 30° C. for 72 h in 5 ml YPD. Yield of the insulin precursor was determined by RP-HPLC of the culture supernatant, and is expressed relative to the yield of a control strain expressing a leader-insulin precursor fusion protein in which the B29 residue is linked to the A1 residue by a mini C-peptide Ala-Ala-Lys. YAP3 is the YAP3 signal sequence.

One example of a new generated insulin precursor is pAK3768. The sequence EEGEPK (SEQ ID NO:2) is the N-terminal extension to the B-chain and alpha2 is the pre-pro-sequence MRFPSIFTAVLFAASSALAAPVNTTTEDETAQIPAEAVIGYSDLEGDFDVAVLPFSNSTNNGL LFINTTIASIAAKEEGVSMAKR (SEQ ID NO:7).

Another example is pAK4053 where TA39 is the pre-pro-sequence MKLKTVRSAVLSSLFASQVLGQPIDDTESNTTSVNLMADDTESRFATNTTLAGGLDVVNLIS MAKR (SEQ ID NO:8).

Further leader sequences used in the examples are alpha-leader with NcoI-site (SEQ ID NO:13) and alpha4-leader with BgIII-site (SEQ ID NO:14).

Table 1 lists the insulin precursors and expression constructs used for the fermentations to produce the insulin precursors. Each construct has been subjected to two or three independent fermentations and analyses, only a very limited number of fermentations was conducted as a single experiment.

TABLE 1
List of insulin precursors and expression construct used for the
expression in S. cerevisiae MT663, including the precursor
yield and the degree of glycosylation of the precursor.

| | | | C-peptide | | Yield | Glycosy- |
|---|---|---|---|---|---|---|
| Exam- | | | (X-Y se- | | Rel. to | lation |
| ple | Leader | Extension | quence) | Insulin precursor | YAK1220 | % |
| YAK | alpha | EEAEAEA | AAK | HIPdesB30 | 1.00 | 0.6 |
| 1220 | | PK | | | | |
| 2 | alpha | EEGEPK | AMK | HIPdesB30 | 1.84 | 0.3 |
| 3 | alpha | EEGEPK | RMK | HIPdesB30 | 1.52 | 0.1 |
| 4 | alpha | EEGEPK | NMK | HIPdesB30 | 1.84 | 0.2 |
| 5 | alpha | EEGEPK | DMK | HIPdesB30 | 2.43 | 0.3 |
| 6 | alpha | EEGEPK | QMK | HIPdesB30 | 1.79 | 0.2 |
| 7 | alpha | EEGEPK | EMK | HIPdesB30 | 1.33 | 0.1 |
| 8 | alpha | EEGEPK | GMK | HIPdesB30 | 1.24 | 0.3 |
| 9 | alpha | EEGEPK | HMK | HIPdesB30 | 1.38 | 0.2 |
| 10 | alpha | EEGEPK | IMK | HIPdesB30 | 1.44 | 0.1 |
| 11 | alpha | EEGEPK | LMK | HIPdesB30 | 1.52 | 0.1 |
| 12 | alpha | EEGEPK | KMK | HIPdesB30 | 1.38 | 0.1 |
| 13 | alpha | EEGEPK | MMK | HIPdesB30 | 1.54 | 0.1 |
| 14 | alpha | EEGEPK | FMK | HIPdesB30 | 0.58 | 0.0 |
| 15 | alpha | EEGEPK | SMK | HIPdesB30 | 1.94 | 0.2 |
| 16 | alpha | EEGEPK | TMK | HIPdesB30 | 2.37 | 0.1 |
| 17 | alpha | EEGEPK | VMK | HIPdesB30 | 1.51 | 0.2 |
| 18 | alpha | no | AMK | HIPdesB30 | 0.43 | 0.0 |
| 19 | alpha | no | DMK | HIPdesB30 | 0.64 | 0.2 |
| 20 | TA39 | EEGEPK | AMK | HIPdesB30 | 1.49 | 0.6 |
| 21 | TA39 | EEGEPK | DMK | HIPdesB30 | 2.44 | 0.7 |
| 22 | alpha | EEGEPK | AMK | IPdesB30*[B28D] | 0.39 | 0.1 |
| 23 | alpha | no | MMK | HIPdesB30 | 0.38 | 0.0 |
| 24 | alpha | no | LMK | HIPdesB30 | 0.39 | 0.0 |
| 25 | alpha | EEAEAEA | AMK | HIPdesB30 | 2.14 | 0.2 |
| | | EPK | | | | |
| 26 | alpha | EEAEAEA | AMK | HIPdesB30 | 1.99 | 0.2 |
| | | PK | | | | |
| 27 | alpha | EEAEPK | AMK | HIPdesB30 | 1.81 | 0.2 |
| 28 | alpha | EEGEPK | ADMK | HIPdesB30 | 1.80 | |
| 29 | alpha | EEGEPK | SDMK | HIPdesB30 | 2.05 | 0.7 |
| 30 | alpha | EEGEPK | TDMK | HIPdesB30 | 2.28 | 0.4 |
| 31 | alpha | EEGEPK | NDMK | HIPdesB30 | 1.70 | |
| 32 | alpha | EEGEPK | GDMK | HIPdesB30 | 2.36 | 0.2 |
| 33 | alpha | EEGEPK | STMK | HIPdesB30 | 1.45 | |
| 34 | alpha | EEGEPK | TTMK | HIPdesB30 | 1.58 | |
| 35 | alpha | EEGEPK | GTMK | HIPdesB30 | 1.89 | |
| 36 | alpha | EEGEPK | TNMK | HIPdesB30 | 1.63 | |
| 37 | alpha | EEGEPK | GNMK | HIPdesB30 | 1.93 | 0.1 |
| 38 | alpha | EEGEPK | SSMK | HIPdesB30 | 1.34 | |
| 39 | alpha | EEGEPK | TSMK | HIPdesB30 | 1.89 | |
| 40 | alpha | EEGEPK | GSMK | HIPdesB30 | 1.79 | 0.0 |
| 41 | TA39 | EEGEPK | NMK | HIPdesB30 | 2.00 | 0.5 |
| 42 | TA39 | EEGEPK | SMK | HIPdesB30 | 1.25 | 0.1 |
| 43 | alpha | EEGEPK | STDMK | HIPdesB30 | 1.65 | |
| 44 | alpha | EEGEPK | DTDMK | HIPdesB30 | 1.46 | |
| 45 | alpha | EEGEPK | AGDMK | HIPdesB30 | 1.11 | |
| 46 | alpha | EEGEPK | SGDMK | HIPdesB30 | 1.59 | |
| 47 | alpha | EEGEPK | TGDMK | HIPdesB30 | 1.34 | |
| 48 | alpha | EEGEPK | DGDMK | HIPdesB30 | 1.41 | |
| 49 | alpha | EEGEPK | AAMK | HIPdesB30 | 1.10 | |
| 50 | alpha | EEGEPK | SAMK | HIPdesB30 | 1.10 | |
| 51 | alpha | EEGEPK | TAMK | HIPdesB30 | 1.41 | |
| 52 | alpha | EEGEPK | GAMK | HIPdesB30 | 1.82 | |
| 53 | alpha | EEGEPK | DAMK | HIPdesB30 | 1.63 | |
| 54 | alpha | EEGEPK | DDAMK | HIPdesB30 | 2.02 | |
| 55 | alpha | EEGEPK | DDMK | HIPdesB30 | 1.89 | |
| 56 | alpha | EEGEPK | ADDMK | HIPdesB30 | 2.03 | |
| 57 | alpha | EEGEPK | SDDMK | HIPdesB30 | 2.43 | |
| 58 | alpha | EEGEPK | TDDMK | HIPdesB30 | 1.30 | |
| 59 | alpha | EEGEPK | GDDMK | HIPdesB30 | 1.46 | |
| 60 | alpha4 | EEGEPK | AMK | HIPdesB30 | 2.01 | |
| 61 | alpha4 | EEGEPK | DMK | HIPdesB30 | 2.31 | |
| 62 | alpha4 | EEAEPK | AMK | HIPdesB30 | 1.99 | |
| 63 | alpha4 | EEAEPK | DMK | HIPdesB30 | 2.50 | |
| 64 | alpha | no | AMK | IPdesB30[B25H_A14E] | 1.61 | |
| 65 | alpha | no | AMK | IPdesB30[B16H_B25H_A14E] | 1.74 | |
| 66 | alpha | no | AMK | IPdesB30[B25H_desB27_A14E] | 1.17 | |
| 67 | alpha | EEGEPK | AMK | IPdesB30[B25H_A14E] | 2.73 | |
| 68 | alpha | EEGEPK | AMK | IPdesB30[B16H_B25H_A14E] | 2.66 | |
| 69 | alpha | EEGEPK | AMK | IPdesB30[B25H_desB27_A14E] | 2.34 | |
| 70 | alpha | EEGEPK | AMK | IPdesB30[desB27_A14E] | 1.65 | |
| 71 | alpha | EEGEPK | DMK | IPdesB30[B25H_A14E] | 3.10 | |
| 72 | alpha | EEGEPK | DMK | IPdesB30[B16H_B25H_A14E] | 2.32 | |
| 73 | alpha | EEGEPK | DMK | IPdesB30[B25H_desB27_A14E] | 2.36 | |
| 74 | alpha | EEGEPK | DMK | IPdesB30[desB27_A14E] | 2.02 | |
| 75 | alpha4 | EEGEPK | SDDMK | HIPdesB30 | 2.77 | |
| 76 | alpha2 | EEGEPK | ADK | HIPdesB30 | 1.64 | 2.1 |
| 77 | alpha2 | EEGEPK | AEK | HIPdesB30 | 1.62 | 2.4 |
| 78 | alpha2 | EEGEPK | AMK | HIPdesB30 | 2.06 | 0.4 |
| 79 | alpha2 | EEGEPK | APK | HIPdesB30 | 1.14 | 2.6 |
| 80 | alpha2 | EEGEPK | AWK | HIPdesB30 | 0.83 | 0.1 |
| 81 | alpha2 | EEGEPK | AAK | HIPdesB30 | 1.10 | 1.1 |
| 82 | alpha2 | EEGEPK | DAK | HIPdesB30 | 1.34 | 1.1 |
| 83 | alpha2 | EEGEPK | EAK | HIPdesB30 | 1.74 | 1.9 |
| 84 | alpha2 | EEGEPK | LAK | HIPdesB30 | 0.73 | 0.2 |

### Example 85

Further insulin precursors and expression constructs were prepared and tested by the same methods as described in Examples 2-84.

Table 2 lists the insulin precursors and expression constructs used for the fermentations to produce the insulin precursors. In the table is also listed the yield and glycosylation level as determined for each construct by duplicate or triple fermentations.

TABLE 2
List of reference insulin precursors and
expression construct used for the expression
in S. cerevisiae MT663, including the precursor
yield and the degree of glycosylation
of the precursor.

| | | | C-pep- | | | |
|---|---|---|---|---|---|---|
| | | | tide | | Yield | Glyco- |
| | | | (X-Y | Insulin | Rel. | sy- |
| Exam- | | Exten- | se- | pre- | to YAK | lation |
| ple | Leader | sion | quence) | cursor | 1220 | % |
| YAK | alpha | EEAEAE | AAK | HIPdesB30 | 1.00 | 0.6 |
| 1220 | | APK | | | | |
| 85A | alpha | no | ALK | HIPdesB30 | 0.21 | |
| 85B | alpha | EEGEPK | AMDK | HIPdesB30 | 0.49 | |
| 85C | alpha | EEGEPK | AMIK | HIPdesB30 | 0.05 | |
| 85D | alpha | EEGEPK | AMTK | HIPdesB30 | 0.19 | |
| 85E | alpha | EEGEPK | AMVK | HIPdesB30 | 0.08 | |
| 85F | alpha2 | EEGEPK | AFK | HIPdesB30 | 0.91 | 0.7 |
| 85G | alpha2 | EEGEPK | AGK | HIPdesB30 | 0.75 | 1.6 |
| 85H | alpha2 | EEGEPK | AKK | HIPdesB30 | 0.93 | 2.5 |
| 851 | alpha2 | EEGEPK | ANK | HIPdesB30 | 1.04 | 2.1 |
| 85J | alpha2 | EEGEPK | FAK | HIPdesB30 | 0.31 | 0.9 |
| 85K | alpha2 | EEGEPK | GAK | HIPdesB30 | 0.80 | 1.1 |
| 85L | alpha2 | EEGEPK | IAK | HIPdesB30 | 0.85 | 0.8 |
| 85M | alpha2 | EEGEPK | PAK | HIPdesB30 | 0.24 | 3.3 |
| 85N | alpha2 | EEGEPK | RAK | HIPdesB30 | 0.64 | 1.4 |
| 85O | alpha2 | EEGEPK | SAK | HIPdesB30 | 0.81 | 1.3 |
| 85P | alpha2 | EEGEPK | WAK | HIPdesB30 | 0.12 | 0.3 |
| 85Q | LA19 | EEAEPK | AAK | HIPdesB30 | 1.30 | 1.21 |
| 85R | TA39 | DDGDPR | DGR | HIPdesB30 | 0.77 | 1.39 |
| 85S | alpha | DDGDPR | DGR | HIPdesB30 | 0.79 | 0.84 |
| 85T | TA57 | EEGEPR | EPR | HIPdesB30 | 1.39 | 2.18 |
| 85U | alpha | EEGEPR | EPR | HIPdesB30 | 1.59 | 1.22 |
| 85V | TA39 | EEGEPR | EPR | HIPdesB30 | 2.47 | 1.03 |

### Example 86-97

To assess the effect of the Y in sequence Z-B-X-Y-A being either K or R, a number of insulin precursors and expression constructs were prepared and tested by the same methods as described in Examples 2-84.

Table 3 lists the insulin precursors and expression constructs used for the fermentations to produce the insulin precursors. In the table is also listed the yield as determined for each construct by duplicate or triple fermentations. It is observed that for the human insulin precursors having the structure Z-B-X-Y-A the yield is on the same level for any of the X-sequences irrespective of the Y-sequence being K (lysine) or R (arginine).

TABLE 3
List of human insulin precursors and
expression construct used for the
expression in S. cerevisiae MT663,
including the precursor yield.

| | | | | C-pep- | |
|---|---|---|---|---|---|
| | | | | tide | Yield |
| | | | Insulin | (X-Y | Rel. |
| Exam- | | Exten- | pre- | se- | to YAK |
| ple | Leader | sion | cursor | quence) | 1220 |
| YAK | alpha | EEAEAE | HIPdesB30 | AAK | 1.00 |
| 1220 | | APK | | | |
| 86 | alpha | EEGEPK | HIPdesB30 | AMK | 1.81 |
| 87 | alpha | EEGEPK | HIPdesB30 | AMR | 1.40 |
| 88 | alpha | EEGEPK | HIPdesB30 | DMK | 2.25 |
| 89 | alpha | EEGEPK | HIPdesB30 | DMR | 1.92 |
| 90 | alpha | EEGEPK | HIPdesB30 | SDDMK | 2.52 |
| 91 | alpha | EEGEPK | HIPdesB30 | SDDMR | 2.42 |

Table 4 lists the insulin aspart precursors and expression constructs used for the fermentations to produce the insulin aspart precursors. In the table is also listed the yield relative to the human insulin precursor as determined for each construct by duplicate or triple fermentations. It is observed that for the insulin aspart precursors having the structure Z-B-X-Y-A the yield is on the same level for any of the X-sequences irrespective of the Y-sequence being K (lysine) or R (arginine). It is noted that the yields of the insulin aspart precursors are normalised against the “reference” human insulin precursor, thus explaining the relative yield of less than 1.0.

TABLE 4
List of insulin aspart precursors and
expression construct used for the
expression in S. cerevisiae MT663,
including the precursor yield.

| | | | | C-pep- | |
|---|---|---|---|---|---|
| | | | | tide | Yield |
| | | | Insulin | (X-Y | Rel. |
| Exam- | | Exten- | pre- | se- | to YAK |
| ple | Leader | sion | cursor | quence) | 1220 |
| YAK | alpha | EEAEAE | HIPdesB30 | AAK | 1.00 |
| 1220 | | APK | | | |
| 92 | alpha | EEGEPK | IPdesB30* | AMK | 0.37 |
| | | | [B28D] | | |
| 93 | alpha | EEGEPK | IPdesB30* | AMR | 0.34 |
| | | | [B28D] | | |
| 94 | alpha | EEGEPK | IPdesB30* | DMK | 0.38 |
| | | | [B28D] | | |
| 95 | alpha | EEGEPK | IPdesB30* | DMR | 0.42 |
| | | | [B28D] | | |
| 96 | alpha | EEGEPK | IPdesB30* | SDDMK | 0.26 |
| | | | [B28D] | | |
| 97 | alpha | EEGEPK | IPdesB30* | SDDMR | 0.24 |
| | | | [B28D] | | |

### Example 98-103

Insulin precursors were tested for level of O-glycosylation as described in example 2 by expression in yeast strains where the gene for either PMT1 or PMT2 has been disrupted by normal yeast genetics methods and compared to insulin precursors expressed in the yeast strain MT663.

The expression constructs were made according to the procedure of example 1 and the fermentation and O-glycosylation analysis were carried out according to the procedures described in examples 2-84.

The insulin precursor expressed in Example 98 having DMK as the C-peptide exhibits a fairly low level of 0.29% O-glycosylation as compared to e.g. the YAK1220 construct in Table 1 (0.6%). Examples 99 and 100 demonstrate that expression of the very same insulin precursor in a Δpmt1 as well as in a Δpmt2 strain lowers the O-glycosylation level even further, i.e. from 0.37% in the wild type strain to 0.17% and 0.13% in the two protein mannosyl transferase knock-out strains, respectively. Hence the O-glycosylation of the insulin precursor is reduced by 2.2 to 2.9 fold by expression in the two different protein mannosyl transferase knock-out strains. The same conclusion follows from the other insulin precursor having the SDMK C-peptide, although here the reduction of O-glycosylation is 3.0 fold in the Δpmt1 strain and 2.4 fold in the Δpmt2 strain.

TABLE 5
Comparison of degree of O-glycosylation
of different insulin precursors
expressed in S. cerevisiae MT663 (wt)
and protein mannosyl transferase
knock-outs strains.

| | | | C-pep- | | | Gly- |
|---|---|---|---|---|---|---|
| | | | tide | | | cosy- |
| | | | (X-Y | | Gene | la- |
| Exam- | | Exten- | se- | Insulin | knock- | tion |
| ple | Leader | sion | quence) | precursor | out | (%) |
| 98 | alpha | EEGEPK | DMK | HIPdesB30 | wt | 0.37 |
| 99 | alpha | EEGEPK | DMK | HIPdesB30 | Δpmt1 | 0.17 |
| 100 | alpha | EEGEPK | DMK | HIPdesB30 | Δpmt2 | 0.13 |
| 101 | alpha | EEGEPK | SDMK | HIPdesB30 | wt | 0.72 |
| 102 | alpha | EEGEPK | SDMK | HIPdesB30 | Δpmt1 | 0.24 |
| 103 | alpha | EEGEPK | SDMK | HIPdesB30 | Δpmt2 | 0.30 |

1. An insulin precursor comprising the sequence Z-B-X-Y-A, wherein
Z is an optional extension sequence,
B is the B-chain of human insulin or an analogue thereof,
X is a sequence selected from the group consisting of X₁M, EA, AE, AD, DA, and AP, where X₁ is a sequence comprising from 1 to 3 amino acid residues,
Y is K or R, and
A is the A-chain of human insulin or an analogue thereof.

2. The insulin precursor according to claim 1, wherein X is X₁M.

3. The insulin precursor according to claim 2, wherein all of the amino acid residues in X₁ are selected from G, A, V, L, I, M, Q, N, E, D, S and T.

4. The insulin precursor according to claim 2, wherein X₁ is selected from D, SDD and A.

5. The insulin precursor according to claim 1, wherein X is selected from EA, AE, AD, DA, and AP.

6. The insulin precursor according to claim 1, wherein Y is K.

7. The insulin precursor according to claim 1, wherein the precursor is human insulin or DesB30-human insulin; and wherein A is A(1-21) and B is B(1-30) or B(1-29).

8. A method for making mature human insulin or an analogue thereof, said method comprising (i) culturing a fungi cell comprising a DNA sequence encoding a precursor for human insulin or an analogue thereof according to claim 1 under suitable culture conditions for expression of said precursor for human insulin or an analogue of human insulin; and (ii) isolating the expressed precursor.

9. A method for reducing O-glycosylation of a precursor for human insulin or an analogue of human insulin during expression in a fungi cell, said method comprising (i) culturing a fungi cell comprising a DNA sequence encoding a precursor for human insulin or an analogue thereof according to claim 1 under suitable culture conditions for expression of said precursor for human insulin or an analogue of human insulin.

10. A method for increasing the yield of a precursor for human insulin or an analogue of human insulin during expression in a fungi cell, said method comprising (i) culturing a fungi cell comprising a DNA sequence encoding a precursor for human insulin or an analogue thereof insulin according to claim 1 under suitable culture conditions for expression of said precursor for human insulin or an analogue of human insulin.

11. The method according to claim 8, wherein said fungi cell carries at least one genetic modification within the genes for PMT1 or PMT2 reducing its capacity for O-glycosylation.

12. The method according to claim 8, wherein said fungi cell is a yeast.

13. The method according to claim 12, wherein said yeast is selected from Saccharomyces cerevisiae, Pichia pastoris and Hansenula polymorpha.

14. An expression vector comprising a polynucleotide sequence encoding a precursor for human insulin or an analogue of human insulin according to claim 1.

15. A host cell transformed with a vector according to claim 14.
